(19) United States
(12) Patent Application Publication
(10) Pub. No.: US 2016/0309821 A1
(43) Pub. Date: Oct. 27, 2016

(54) METHOD FOR ELIMINATING BALDNESS USING HAIR FRAGMENTS AND ADHESIVE

(71) Applicant: Ryan KNIGHT, Perris, CA (US)

(72) Inventor: Ryan KNIGHT, Perris, CA (US)

(21) Appl. No.: 15/104,958

(22) PCT Filed: Dec. 19, 2014
(86) PCT No.: PCT/US2014/071712

§ 371 Date: Jun. 15, 2016

Related U.S. Application Data
(60) Provisional application No. 61/918,044, filed on Dec. 19, 2013.

(51) Int. Cl. A41G 5/00; A61Q 5/00; A61K 8/98

(52) U.S. Cl. CPC A41G 5/0033; A61K 8/985; A61Q 5/00; A61K 2800/80

(57) ABSTRACT

A method for eliminating baldness and recreating the hairline of a natural-looking low cut hair style, by adhering short fragments of hair onto the scalp with an adhesive. The method includes drawing a desired hairline shape on the scalp (if needed), applying an adhesive to the desired areas using the drawn hairline, and sprinkling the hair fragments onto the adhesive, such that the hair fragments are affixed onto the scalp. Various tools may be used to help draw the hairline and to shape the hairline afterwards. The hair fragments can be removed using a removal agent that dissolves the adhesive. The method is relatively simple once learned, easy to apply, and creates immediate results.

### BACKGROUND OF THE INVENTION

1. Field of the Invention

This invention relates to a method for eliminating baldness and restoring the hairline for bald individuals by adhesively affixing hair fragments onto the scalp.

2. Description of the Related Art

Various hair restoration or hair replacement methods are available for individuals with hair loss problems, including hair transplant surgery and non-surgical methods such as by using laser or medications. These methods are often complex and expensive.

### SUMMARY OF THE INVENTION

The present invention is directed to a method and related tools for eliminating baldness and restoring the hairline in individuals with baldness problems, both male and female, but particularly suitable for males. By adhering short fragments of hair onto the scalp with adhesive, it creates a low cut hair style that is virtually indistinguishable from natural hair, with immediate results.

An object of the present invention is to provide an easy to use method for creating natural-looking low cut hair style for individuals with baldness problems.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve these and other advantages and in accordance with the purpose of the present invention, as embodied and broadly described, the present invention provides a method for eliminating baldness and recreating the hairline on a scalp, which includes: applying an adhesive to defined areas of the scalp; and sprinkling short fragments of hair onto the adhesive, wherein the fragments of hair have lengths of less than ¼ inches. Preferably, the fragments of hair have lengths of less than 1/16 inches.

It is to be understood that both the foregoing general description and the following detailed description are exemplary and explanatory and are intended to provide further explanation of the invention as claimed.

### BRIEF DESCRIPTION OF THE DRAWINGS

FIG. 1 schematically illustrates a method for eliminating baldness and recreating the hairline according to an embodiment of the present invention.

FIGS. 2A and 2B illustrate the head of a bald individual before and after applying the method.

### DETAILED DESCRIPTION OF THE PREFERRED EMBODIMENTS

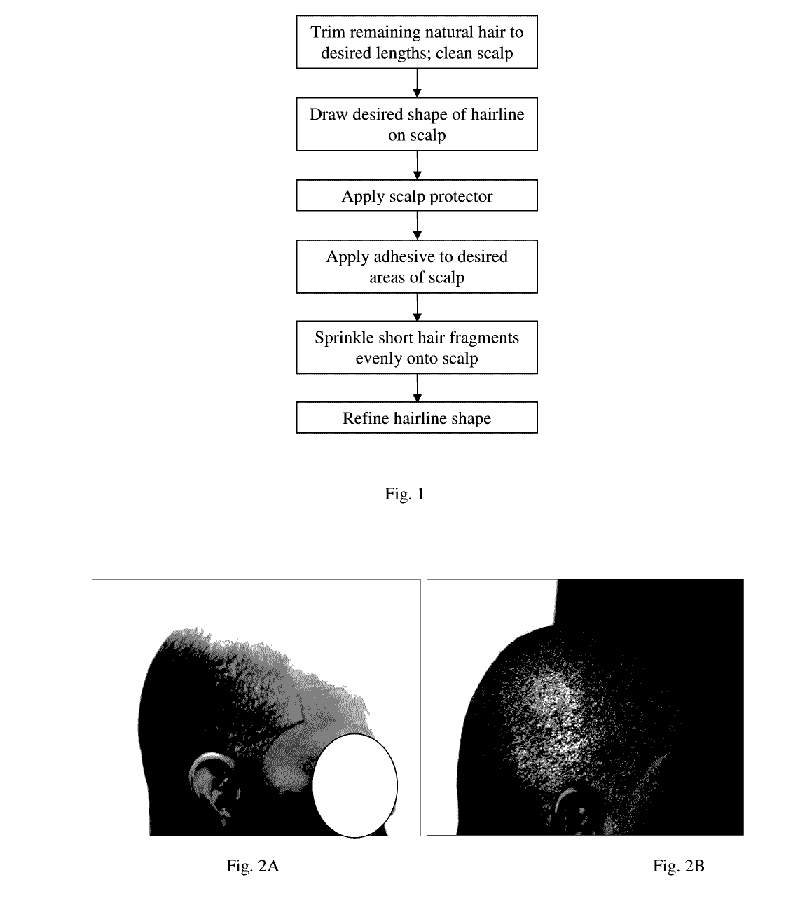

The hairline restoration method according to an embodiment of the present invention includes the following steps (see FIG. 1).

First, the remaining natural hair is trimmed to desired lengths, e.g., with a hair clipper. The scalp is cleaned (e.g., with alcohol wipes). A scalp protector may be applied if desired. After the scalp protector dries, an adhesive is applied to desired areas of the scalp. Prior to applying the scalp protector and the adhesive, a desired shape of the hairline may be drawn on the scalp to guide the adhesive application (optional). If the individual has a bald area surrounded by remaining hair, then it is not necessary to draw the hairline. Then, short hair fragments (preferably 1/16 of an inch or shorter) are sprinkled evenly onto the scalp and adhered to the scalp by the adhesive. The amount of hair fragment used may depend on individual preference. Before the adhesive dries, the hairline shape formed by the hair fragments may be refined by using hairline sculpting tools to push or otherwise shape the hair and adhesive at the boundaries. Once the adhesive dries, the hair fragments become securely affixed to the scalp by the adhesive. Parts of the hair fragments will be embedded in the adhesive and the rest are exposed, forming a natural looking hair texture. Once the hair fragments are affixed to the scalp, the hair can withstand physical workout, rain, swimming, showers, etc. The affixed hair may be removed using a remover solution that dissolves the adhesive, coupled with shampoo.

FIGS. 2A and 2B show the head of a bald individual before and after applying the above-described process. FIG. 2 shows that the baldness is covered and a hairline is created.

In this process, the hair may be referred to as being “fused” onto the scalp, and the process may be referred to as “hair fusion.” This process is effective both for hair with small curls and for more straight hair.

Any type of hair may be used to make the hair fragments, including natural hair and synthetic hair, or any fragments that resemble hair. For example, in one embodiment, the hair fragments used are synthetic (1B) Vienna Hair Collection B. Marley pony hair. Some examples of commercially available hair that can be used are: Model Model Glance Twist (1B); Vienna Hair Collection, B. Marley Pony (1B); Premium Synthetic Hair Afro Marley Braid (1B); etc. The color of the hair will depend on users' individual preferences.

The hair may be cut into desired lengths manually. Alternatively, suitable machines may be used to cut large quantities of hair, e.g. on a commercial basis. The desired lengths of the hair fragments may depend upon the type of style that the user is trying to achieve. In one example, for African American hair styles, the hair is cut to 1/16 of an inch or shorter. Other sizes may also be used, but generally no longer than ¼ inches. Here, for curly hair, hair fragment length refers to the dimension of the hair when curled up, not the linear length when it is straightened. The hair fragments used may have a distribution of lengths to create a natural appearance.

The adhesive used in the hair fusion process is preferably a liquid adhesive. Any suitable, skin-safe adhesive may be used. In one example, the adhesive is Ben Nye Spirit Gum, which works well for an African American look. Some examples of commercially available adhesives that can be used include: Ben Nye spirit gum, Merhon spirit gum, Krylon Sprit gum, Davlyn Super Stick Adhesive, Mity Tight Acrylic Adhesive, Max Dermatex Adhesive, etc. The compositions of these adhesives are generally known and are not described in detail here. The adhesive may be applied with a brush directly to areas of the scalp that the user would like to place hair. Preferably, it is applied with long even strokes back and forth or side to side. The adhesive should be evenly distributed over the desired areas. The amount of adhesive applied to the head will be dictated by the desired style and look.

A scalp protector may be optionally applied to the clean scalp and allowed to dry until it is not tacky to touch, before the adhesive is applied. The main purpose of the scalp protector is to form a barrier to protect the skin from irritation that might be caused by the adhesive. Also, oily skin may weaken the adhesive hold, especially in hot and humid weather. Some examples of commercially available scalp protectors that can be used are: Walkers skin protector, Brandywine Skin protector, Davlyn skin protector, etc. The compositions of these products are generally known and are not described in detail here.

Prior to applying the adhesive and scalp protector, various tools may be used to draw a desired shape of the hairline on the scalp to guide the adhesive application. This step is optional. The purpose is to draw a visible hairline that will allow the user to know where the borders of the hairline should be to start and stop the placement of adhesive. An eye line pencil or other drawing tools may be used. Other tools may be used to create desired straight or curved lines for the hairline, such as: a headband, used as a forehead hairline tracer tool, which can be placed around the head to help create an evenly drawn horizontal hairline on the forehead; a protractor, used as a temple hairline tracer, which can be placed at the side of the temple and traced to help create the arc of the hairline on the temples and to connect them with the forehead hairline; a tape measure, which can be used to measure the distances of the various lines on the sides to make sure that they are symmetrical; etc. It is also possible to use 3-D imaging and 3-D printing techniques to fabricate custom-made head masks for individual users, which can be used to draw the hairlines with ease.

When sprinkling hair fragments on to the head after applying the adhesive, the hair should be sprinkled evenly over the desired areas. This can be done manually using fingers. Optionally, a sifter or similar tool may be used to evenly apply hair fragments onto the head.

After applying the adhesive and sprinkling the hair onto the head, and before the adhesive dries, the user may pat all sides, top and back of the head with the open palms of the hands to make sure that the hair will mesh onto the coat of adhesive, and the hair that does not will fall off. In addition, the hairline can be shaped by pushing the hair along the hairline using tools of various shapes, such as elongated plates with straight or curved edges. These tools can be used for a final touch styling, to make sure that the hair lines are crisp and sharp and no hairs are out of place. Optionally, a clipper may be used to cut hairs that stick up or out, or to cut the hair lower if desired, etc.

Optionally, after the hair fusion process described above, a color spray, such as Black Ice Chrometome spray, may be used to add colored coverage to areas that may not have received as much hair as desired or if the user wants to have a darker hair color.

When the user wishes to remove the affixed hair, a hairline removal liquid may be used to dissolve the adhesive. The hairline removal liquid is applied to the hair, either by using the hands or by spraying the removal solution onto the head. Preferably, it is allowed to saturate the hair for a period of time, e.g. 15 to 25 minutes or longer, for easier hair removal. A comb such as a rat tail comb can be used to removal the hair by combing out, and a shampoo can be used to wash off the hair. Various substances or mixtures may be used as the hairline removal liquid, for example: a mixture of ⅓ Citrus 22 solvent, ⅓ cooking oil (e.g. Wesson), and ⅓ Olive oil; Citrus 22 solvent; Max Adhesive remover; Merhon spirit gum remover; Pure Adhesive remover; Olive oil; etc. The compositions of these products are generally known and are not described in detail here. The suitable removal agents may depend on the adhesive used.

A kit containing various materials and tools needed for the above described process may be provided as a commercial product to allow individual users to practice the hairline restoration method themselves. The kit may include, for example: package of hair fragments, adhesive, removal solution, the various tools described earlier, etc.

This process can be used to recreate the hairline, cover up bald areas and/or scars, keloid, etc. Once the scalp is prepped, baldness can be eliminated within an hour.

It will be apparent to those skilled in the art that various modification and variations can be made in the baldness elimination and hairline restoration method of the present invention without departing from the spirit or scope of the invention. Thus, it is intended that the present invention cover modifications and variations that come within the scope of the appended claims and their equivalents.

What is claimed is:

1. A method for eliminating baldness and recreating a hairline on a scalp, comprising:
applying an adhesive to defined areas of the scalp; and
sprinkling short fragments of hair onto the adhesive, wherein the fragments of hair have lengths of less than ¼ inches.

2. The method of claim 1, wherein the fragments of hair have lengths of less than 1/16 inches.

3. The method of claim 1, wherein the fragments of hair are made of natural hair or synthetic hair.

4. The method of claim 1, wherein the adhesive is Ben Nye spirit gum, Merhon spirit gum, Krylon Sprit gum, Davlyn Super Stick Adhesive, Mity Tight Acrylic Adhesive, or Max Dermatex Adhesive.

5. The method of claim 1, further comprising: prior to applying the adhesive, drawing an outline of the defined areas where the adhesive is to be applied.

6. The method of claim 1, further comprising: prior to applying the adhesive, applying a scalp protector to the scalp, wherein the adhesive is applied after the scalp protector dries.

7. The method of claim 1, further comprising: after sprinkling the hair fragments and before the adhesive dries, shaping a boundary of the adhesive by pushing it with a tool.

8. The method of claim 1, further comprising: removing the hair fragments from the scalp by applying a removal agent to dissolve the adhesive.

9. The method of claim 8, wherein the removal agent is Citrus 22 solvent, Max Adhesive remover, Merhon spirit gum remover, Pure Adhesive remover, olive oil, or a mixture thereof.
